(19) United States
(12) Patent Application Publication
(10) Pub. No.: US 2013/0029384 A1
(43) Pub. Date: Jan. 31, 2013

(54) THERMOSTABLE SUCROSE PHOSPHORYLASE

(71) Applicants: An Cerdobbel, Merelbeke (BE); Tom Desmet, Nevele (BE); Wim Soetaert, Lovendegem (BE)

(72) Inventors: An Cerdobbel, Merelbeke (BE); Tom Desmet, Nevele (BE); Wim Soetaert, Lovendegem (BE)

(73) Assignee: UNIVERSITEIT GENT, Gent (BE)

(21) Appl. No.: 13/639,321

(22) PCT Filed: Apr. 1, 2011
(86) PCT No.: PCT/EP11/55139

§ 371 Date: Oct. 4, 2012

(30) Foreign Application Priority Data
Apr. 6, 2010 (GB) 1005754.5
Jun. 30, 2010 (EP) 10167989.2
Dec. 9, 2010 (EP) 10194409.8
Feb. 14, 2011 (EP) 11154339.3

(51) Int. Cl. C12N 9/10; C12N 15/70; C12N 11/10; C12P 19/02; C12N 11/16; C12N 11/08

(52) U.S. Cl. 435/105; 435/193; 435/174; 435/180; 435/178; 435/471

(57) ABSTRACT

The present invention relates to a sucrose phosphorylase from Bifidobacterium adolescentis which is useful as a biocatalyst in carbohydrate conversions at high temperatures. Indeed, the biocatalysts of the present invention are enzymatically active for a time period of at least 16 h and up to 1 to 2 week(s) at a temperature of at least 60° C. The biocatalysts of the present invention are: a) immobilized on an enzyme carrier, or b) are part of a cross-linked enzyme aggregate (CLEA), and/or c) are mutated, and/or d) are enzymatically active in the continuous presence of their substrate.

### TECHNICAL FIELD OF INVENTION

The present invention relates to a sucrose phosphorylase from Bifidobacterium adolescentis which is useful as a biocatalyst in carbohydrate conversions at high temperatures. Indeed, the biocatalysts of the present invention are enzymatically active for a time period of at least 16 h and up to 1 to 2 week(s) at a temperature of at least 60° C. The biocatalysts of the present invention are: a) immobilized on an enzyme carrier, or b) are part of a cross-linked enzyme aggregate (CLEA), and/or c) are mutated, and/or d) are enzymatically active in the continuous presence of their substrate.

### BACKGROUND ART

Sucrose phosphorylase (SPase) catalyses the reversible phosphorolysis of sucrose into α-D-glucose-1-phosphate (α-D-G1P) and fructose. This enzyme is mainly found in lactic acid bacteria and bifidobacteria, where it contributes to an efficient energy metabolism (Lee et al., 2006). Indeed, the produced glycosyl phosphate can be catabolised through glycolysis without further activation by a kinase, which results in the saving of one ATP molecule compared to the action of a hydrolytic enzyme. SPase is formally classified as a glycosyl transferase (EC 2.4.1.7), although it belongs to glycoside hydrolase family 13 (Henrissat, 1991) and follows the typical double displacement mechanism of retaining glycosidases (Goedl and Nidetzky, 2009). The crystal structure of the enzyme from Bifidobacterium adolescentis has been determined, and its catalytic aminoacids have been shown to be Asp192 (nucleophile) and Glu232 (acid/base) (Sprogoe et al., 2004).

A number of practical applications have been developed for SPase (Birnberg and Brenner, 1984; Tedokon et al., 1992). Because of the broad acceptor specificity of the enzyme, it can be used for the production of α-D-G1P (Goedl et al., 2007) as well as a number of glycosylated compounds (Kitao et al., 1995). At the industrial scale, such carbohydrate conversions are preferably run at 60° C. or higher, mainly to avoid microbial contamination. Unfortunately, no such SPase enzymes have yet been identified in thermophilic organisms. The highest temperature optimum reported so far is 48° C. for the SPase enzyme from B. adolescentis (van den Broek et al., 2004).

The thermostability of an enzyme can be further increased in several ways, most notably by means of mutagenesis or immobilization (Unsworth et al., 2007). The thermostability of the SPase from Streptococcus mutans, for example, has been increased about 20-fold by the introduction of 8 amino acid mutations (Fujii et al., 2006). Also US 2008/0206822 to Fujii et al. disclose mutation-based methods for improving the thermostability of SPases derived from Streptococcus mutans, Streptococcus pneumonia, Streptococcus sorbinus, Streptococcus mitis, Leuconostoc mesenteroides, Oenococcus oeni, Lactobacillus acidophilus and Listeria monocytogenes. However, the stability of these enzyme variants is still too low to allow their exploitation in an industrial process. Alternatively, covalent immobilization has been shown to be a very efficient strategy for the rigidification of enzymes. For that purpose, epoxy-activated synthetic carriers such as sepabeads are especially useful (Hilterhaus et al., 2008; Katchalski-Katzir and Kraemer, 2000; Mateo et al., 2007). The epoxy-activated supports are able to react with different nucleophilic groups at a protein's surface (Lys, His, Cys, Tyr etc.), generating a strong covalent attachment. In this regard, Lopez-Gallego et al. (J. Biotech 2004:219) disclose an acylase which is immobilized using amino-epoxy Sepabead and which shows an activity decrease of 30% after 10 h at 45° C. An alternative method for enzyme immobilization, based on the formation of a cross-linked enzyme aggregate (CLEA), has been described by Sheldon et al. (Biocatal Biotransformation 2005). For example, Zhao et al. (J. Mol. Catalysis 2008:7) disclose a Pseudomonas lipase immobilized using the CLEA technology which looses 28% of its activity after 24 h at 60° C. On the other hand, Pimentel and Ferreira (Appl. Biochem. Biotechnol. 1991: 37) demonstrated that no improvement of thermal stability was obtained when an SPase from Leuconostoc mesenteroides was immobilized by covalent linkage to several supports.

It is thus still unclear how, if and/or to what extent the thermal stability of a particular enzyme can be improved. In this regard and for example, it is indicated in WO 2008/034158 that no specific method of immobilization can be chosen for a particular enzyme with the expectation that the immobilization will be successful. It is further generally agreed that a successful immobilization of any enzyme must be discovered by screening a variety of methods, and an optimal result obtained by extensive experimentation.

However, there is a further need to develop biocatalysts such as SPase enzymes which are useful for application in carbohydrate conversions at high temperatures as required by industry. The present invention discloses biocatalysts which comprise a sucrose phosphorylase from Bifidobacterium adolescentis or variants (mutants) thereof and which demonstrate surprisingly favorable properties for use in industry. For example, they retain most of their enzymatic activity at temperatures of 60° C. and higher and/or they even show no activity loss after two weeks of continuous activity in the presence of their substrate. In the case of cross-linked enzyme aggregates (CLEAs), the temperature optimum of the sucrose phosphorylase of the present invention was 17° C. higher than that of the soluble enzyme. Furthermore, the CLEA immobilized enzyme displays an exceptional thermal and operational stability, retaining all of its activity after one week incubation at 60° C. Recycling of said biocatalyst allows its use in at least ten consecutive reactions, which dramatically increases the commercial potential of its glycosylating activity.

### BRIEF DESCRIPTION OF FIGURES

FIG. 1. Effect of temperature on the stability of purified SPase: incubation at 60° C. (), 65° C. (▾), 70° C. (∘) and 75° C. (×) in 0,1M phosphate buffer, pH 6.5 using 0.46 U ml⁻¹ SPase in the reaction mixture.

FIG. 2. Effect of SPase concentration on the thermostability of purified (black) and crude (gray) enzyme preparation: 30 min incubation at 70° C. in 0.1M phosphate buffer, pH 6.5 using different SPase concentration in the reaction mixture.

FIG. 3. Loading capacity of Sepabeads EC-HFA and EC-EP. Enzymatic activity of Sepabeads EC-HFA (∘) and EC-EP () with different loads of enzyme. Actively bound enzyme of Sepabeads EC-HFA (□) and EC-EP (▪).

FIG. 4. The effect of pH on the activity of free (∘) and immobilized SPase () from B. adolescentis.

FIG. 5. Thermoactivity of the free (∘) and immobilized () SPase from B. adolescentis.

FIG. 6. Effect of the SPase concentration on the thermostability. Residual activity of free (▾) and immobilized enzyme, immobilized in absence () or presence of sucrose (∘) after 16 h incubation at 60° C.

FIG. 7. Sepabeads EC-HFA

FIG. 8 General scheme for the production of cross-linked enzyme aggregates (CLEAs).

FIG. 9 The effect of the cross-linking ratio () and reaction time (∘) on the immobilization yield of SPase from B. adolescentis. The immobilization yield is defined as the ratio of the activity detected in the CLEA preparation and that present in the original enzyme solution.

FIG. 10 The effect of pH on the activity of soluble (∘) and immobilized () SPase from B. adolescentis. Reactions were performed with 0.1 M sucrose in a 0.1 M phosphate buffer at 37° C.

FIG. 11 The effect of temperature on the activity of soluble (∘) and immobilized () SPase from B. adolescentis. Reactions were performed with 0.1 M sucrose in a 0.1 M phosphate buffer at pH 7.

### DESCRIPTION OF INVENTION

The present invention relates to a biocatalyst comprising a sucrose phosphorylase from Bifidobacterium adolescentis characterized in that said sucrose phosphorylase is enzymatically active: 1) for a time period of at least 16 h (when mutated and/or when immobilized on a carrier), 2) for at least one week (as part of a CLEA), and/or 3) continuously for a period of at least two weeks in the presence of their substrate, at a temperature of at least 60° C. Hence, the sucrose phosphorylase of the present invention is mutated and/or is immobilized and/or is in the continuous presence of its substrate. Preferably, said sucrose phosphorylase of the present invention is: 1) immobilized on a carrier, such as an epoxy-activated enzyme carrier, or 2) immobilized by cross-linking, e.g. said sucrose phosphorylase of the present invention is part of a cross-linked enzyme aggregate (CLEA), and/or 3) is mutated at specific residues, and/or 4) is in the continuous presence of its substrate. Alternatively, said sucrose phosphorylase is immobilized by entrapment of the sucrose phosphorylase, such as by inclusion (e.g. inclusion bodies).

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

With the term ‘biocatalyst’ is meant an enzyme, in particular a sucrose phosphorylase from Bifidobacterium adolescentis, which is immobilized, e.g. bound to a carrier, preferably covalently bound, and which initiates or modifies the rate of a biochemical reaction. The latter ‘biochemical reaction’ refers to any conversion of a carbohydrate. An example of such a conversion is the breakdown of a disaccharide with the help of inorganic phosphate resulting in a free monosaccharide and a C1-phosphorylated monosaccharide such as the reversible phosphorolysis of sucrose into alpha-D-glucose-1-phosphate and fructose. Because of the reversibility of the latter phosphorolysis, the biocatalyst of the present invention can also be used for the synthesis of glycosidic bounds. As such, and because the biocatalyst of the present invention displays a broad activity towards a variety of carbohydrate and non-carbohydrate acceptors, the biocatalyst of the present invention allows for the synthesis of the corresponding oligosaccharides and glycosides, respectively (Goedl et al. Biocat Biotrans 2010: 10). Important examples of non-carbohydrate acceptors include aliphatic, aromatic and sugar alcohols, ascorbic and kojic acid, furanones and catechins. Even a carboxyl group (e.g. acetic and caffeic acid) can be used as point of attachment, resulting in an ester instead of an ether bond. Recently, an extremely efficient process for the production of 2-O-(α-D-glucopyranosyl)-sn-glycerol has been described (Goedl et al. Angew Chem Int Ed Engl 2008: 10086 and W02008/034158). Under the right conditions, the competing hydrolytic reaction could be completely suppressed, resulting in near quantitative yields. The latter product can be used as moisturizing agent in cosmetic formulations.

The term ‘a sucrose phosphorylase from Bifidobacterium adolescentis’ refers to a protein encoded by a sucrose phosphorylase gene from Bifidobacterium adolescentis and specifically refers to the enzyme as described by Sprogoe et al. (2004). More specifically, the latter term refers to a sucrose phosphorylase encoded by the sucrose phosphorylase gene from Bifidobacterium adolescentis LMG 10502 as described by Reuter (1963) and which is synonymous to DSM20083 and ATTC15703.

The present invention thus relates to a biocatalyst comprising a sucrose phosphorylase from Bifidobacterium adolescentis characterized in that said sucrose phosphorylase is enzymatically active for a time period of at least 16 h at a temperature of at least 60° C. Said sucrose phosphorylase of the present invention is preferably immobilized and/or mutated and/or in the continuous presence of its substrate. In one aspect of the invention, said phosphorylase is immobilized on an epoxy-activated enzyme carrier or is part of a cross-linked enzyme aggregate (CLEA). Said sucrose phosphorylase is, in one aspect of the invention, encoded by the sucrose phosphorylase gene from Bifidobacterium adolescentis LMG 10502. The sucrose phosphorylase of the present invention may further contain at least one mutation (i.e. a deletion, substitution or addition, or any combination thereof) which increases its stability and which does not diminish the sucrose phosphorylase activity by at most 5%, 10% or 20%, preferably by at most 30%, more preferably by at most 40% and most preferably by at most 50%. In other words, the sucrose phosphorylase of the present invention may further contain at least one deletion, substitution or addition, or any combination thereof, in which the sucrose phosphorylase activity is retained by at least 50%, 60%, 70%, 80%, 90%, or even 100%. In a further specific embodiment, the sucrose phosphorylase of the present invention may thus further contain at least one deletion, substitution or addition, or any combination thereof, which does not diminish the sucrose phosphorylase activity by at most 50%. Phosphorylase activity can be measured by any method known in the art such as the coupled enzymatic assay as described by Koga et al. (1991) and Silverstein et al. (1967) or the discontinuous Bicinchonic Acid assay as described by Waffenschmidt and Jaenicke (1987). An example of an addition that does not influence the enzyme's activity is an N-terminal fusion to the enzyme of a peptide with the sequence Gly-Gly-Ser-His6-Gly-Met-Ala-Ser (=a His-tag) for purification purposes. A skilled person understands that other additions such as C-terminal affinity tags or other affinity tags, or deletions or substitutions, preferably conservative substitutions, or any combination thereof that do not diminish the sucrose phosphorylase activity by at most 5%, 10% or 20%, preferably by at most 30%, more preferably by at most 40% and most preferably by at most 50% are further embodiments of the present invention. The present invention further relates to a biocatalyst as defined above, wherein said sucrose phosphorylase contains the following mutations (i.e. substitutions): Q331E, R393N, Q460E/E485H, D445P/D446G, D445P/D446T, R393N/Q460E/E485H, R393N/Q460E/E485H/D445P/D446T, R393N/Q460E/E485H/D445P/D446T/Q331E. The latter enzyme mutants are found to have an increased residual activity and stability for at least 16 compared to the wild-type enzyme. The mutant or enzyme variant containing the mutations R393N/Q460E/E485H/D445P/D446T/Q331E is completely stable (i.e. there is no loss of activity) for at least 16 h at 60° C.

The terms ‘enzymatically active for a time period of at least 16 h at a temperature of at least 60° C.’ refers to a constant and measurable phosphorylase activity—using well known methods—for a time period of 16, 17, 18, 19, 20, 21, 22, 23, 24 hours or 2, 3, 4, 5, 6, 7, 8, 9, 10, 11, 12, 13, 14, 15, 16 or more days and this constantly at a temperature of 60, 61, 62, 63, 64, 65, 66, 67, 68, 69, 70, 71, 72, 73, 74, 75 C.° or higher temperatures. The term ‘active’ refers to ‘fully active’ or ‘retaining 100% of its activity’ or ‘loosing no activity’ when compared to the activity of the soluble, free, native, wild-type enzyme or when compared to the enzyme's activity in the absence of a continuous presence of its substrate, or, refers to a diminished sucrose phosphorylase activity by at most 50% (i.e. a diminished activity of 5%, 10%, 15%, 20%, 25%, 30%, 35%, 40%, 45%, 50% when compared to the activity of the soluble, free, native, wild-type enzyme or when compared to the enzyme's activity in the absence of a continuous presence of its substrate). The term ‘an epoxy-activated carrier’ refers to an enzyme carrier which is capable of covalently binding—and thus immobilizing—enzymes and which comprises a group having the formula R—O—R′, wherein R is any porous matrix material, preferably a highly porous methacrylic polymer matrix spherical bead, and wherein R′ contains at least an epoxy group, i.e. a group consisting of an oxygen atom joined by single bounds to two adjacent carbon atoms thus forming the three-membered epoxy ring. In a particular embodiment, the present invention relates to a biocatalyst as defined above, wherein said amino-epoxy-containing enzyme carrier comprises—as a functional group—the structure as shown in FIG. 7.

In a more particular and preferred embodiment, the present invention relates to a biocatalyst as defined above, wherein said epoxy-activated carrier is Sepabead EC-HFA™.

The term ‘is part of a cross-linked enzyme aggregate (CLEA)’ basically refers to an immobilized enzyme preparation that does not contain a carrier as described by Sheldon et al. (2005). Consequently, the enzyme's activity is not diluted but is still highly stabilized. CLEA's can be prepared by: 1) precipitating the enzyme, 2) cross-linking the enzyme precipitate via adding for example a glutaraldehyde solution and stiffing this mixture, 3) further reducing the cross-linked enzyme via adding for example sodium bicarbonate buffer and sodium borohydride, and finally 4) centrifuging and washing the resulted CLEA.

The present invention further relates to the usage of a biocatalyst as defined above in order to convert carbohydrates as defined above at elevated temperatures as required by industry. As such the biocatalysts of the present invention provide a number of process advantages such as reduced risk of microbial contamination, lower viscosity, improved conversion rates and improved substrate and product solubility. With the term ‘elevated temperatures’ are meant temperatures of about at least 60° C. or more: i.e. 58, 59, 60, 61, 62, 63, 64, 65, 66, 67, 68, 69, 70, 71, 72, 73, 74, 75° C. or even higher temperatures. In particular embodiments, the present invention relates to the usage as defined above wherein said temperature is at least 60° C. and preferably at least 65° C. In other words, the present invention relates to a process to convert carbohydrates as defined above with a biocatalyst as defined above wherein said process comprises at least the step of contacting the enzyme of the present invention with its substrate at a temperature of at least 60° C. and preferably at least 65° C.

A further preferred embodiment of the present invention relates to the usage or process as defined above wherein said conversion of carbohydrates is the phosphorolysis of sucrose into alpha-D-glucose-1-phosphate and fructose and this at a temperature of at least 60° C. and preferably at least 65° C.

In another embodiment, the present invention relates to a process to convert carbohydrates as defined above with a biocatalyst as defined above or to the usage or process as defined above wherein said conversion of carbohydrates is the phosphorolysis of sucrose into alpha-D-glucose-1-phosphate and fructose and this at a temperature of at least 60° C. and preferably at least 65° C. and wherein the enzymes' substrate such is continuously present. For example, the present invention relates to the usage of the catalysts of the present invention as defined above in the continuous presence of sucrose in order to continuously synthesize glucose-1-phosphate at a temperature of at least 60° C.—and preferably at least 65° C.—during a period of at least ¼, ½, 1, 2, 3, 4, 5, 6, 7, 8, 9, 10, 11, 12, 13, 14, 15, 16, or even more day(s).

In another embodiment, the present invention relates to a process to convert carbohydrates as defined above with a biocatalyst as defined above (CLEA) or to the usage or process as defined above wherein said conversion of carbohydrates is the phosphorolysis of sucrose into alpha-D-glucose-1-phosphate and fructose and this at a temperature of at least 60° C. and preferably of at least 65° C.

The present invention further relates to a method to produce the biocatalysts of the present invention comprising the following steps:
purifying a sucrose phosphorylase from Bifidobacterium adolescentis as defined above in order to obtain a solution of purified sucrose phosphorylase,
adding an epoxy-activated carrier as defined above to said solution of purified sucrose phosphorylase in order to obtain an immobilized sucrose phosphorylase,
washing said immobilized sucrose phosphorylase,
inactivating the remaining epoxy-groups on said epoxy-activated carrier which are not linked to a sucrose phosphorylase, and
washing said immobilized sucrose phosphorylase.

The purification step can be undertaken by any method known to a skilled person such as for example purifying by affinity chromatography on a nickel-nitrilotriacetic acid metal matrix. The latter method might be chosen when—for example—the enzyme to be purified is produced in a recombinant manner Hence, the present invention further discloses a method as defined above wherein all steps are preceded by the step of transforming a host organism with a gene encoding for a sucrose phosphorylase from Bifidobacterium adolescentis as defined above and expressing said phosphorylase in said host organism. As an example, the vector in order to clone the above-defined sucrose phosphorylase into can be the vector pCXh34 vector and a host organism can be E. coli XL10-Gold as described by (Aerts et al., 2010). The step of ‘adding an epoxy-activated carrier as defined above to said solution of purified sucrose phosphorylase in order to obtain an immobilized sucrose phosphorylase’, or—in other words—the immobilization step, can be optimized using any optimization methods such as full factorial design as it is known that many factors can influence the efficiency of an immobilization process, including the temperature, pH and ionic strength of the immobilization buffer. Preferentially, the immobilization step is undertaken at 25° C. and the immobilization buffer has a pH of 7.15 and contains a phosphate concentration of 0.04 M.

Hence, the present invention further relates to a method as defined above, wherein the step of adding an epoxy-activated carrier to a solution of purified sucrose phosphorylase are undertaken in an immobilization buffer having a pH of 7.15 and containing a phosphate concentration of 0.04 M.

The washing step as described above can for example be undertaken with 100 mM phosphate buffer. The step ‘inactivating the remaining epoxy-groups on said epoxy-activated carrier which are not linked to a sucrose phosphorylase’ can, for example, be undertaken via treating the immobilized support for 24 h with 5 ml of 3 M glycine solution at pH 8.5 (25° C.). The immobilized sucrose phosphorylase can then be washed with an excess of 100 mM phosphate buffer.

The present invention further relates to a method to produce the biocatalysts of the present invention comprising the following steps:
purifying a sucrose phosphorylase from Bifidobacterium adolescentis as defined above in order to obtain a solution of purified sucrose phosphorylase,
aggregating the enzymes, which can e.g. be achieved by the addition of salts, organic solvents or non-ionic polymers
chemically cross-linking the aggregated enzyme molecules to obtain an immobilised biocatalyst.

The present invention will now be illustrated by the following non-limiting examples.

### EXAMPLES

### Example 1

### Immobilizing the SPase Via the Epoxy-Activated Enzyme Carrier or the CLEA Technology

Materials and Methods

Materials for Immobilization of SPase

Amino-epoxy (EC-HFA) Sepabeads supports were kindly provided by Resindion S.R.L (Mitsubishi Chemical Corporation).

Materials for CLEAs

Tert-butyl alcohol, glutaraldehyde, and sodium borohydride were purchased from Aldrich-Chemie, Fisher and Acros, respectively. All other reagents were purchased from Sigma-Aldrich.

Expression and Purification of SPase

E. coli XL10-Gold cells transformed with the constitutive expression plasmid pCXshP34_BaSP were cultivated in 1-1 shaken flasks at 37° C. containing LB medium supplemented with 100 mg 1⁻¹ ampicillin. After 8 h of expression, the cells were harvested by centrifugation (7000 rpm, 4° C., 20 min), suspended in lysis buffer NPI-10 (Qiagen) and disrupted by sonication. Cell debris was removed by centrifugation (12 000 rpm, 4° C., 30 min) The N-terminal 6-His tagged protein was purified by nickel-nitrilotriacetic acid (Ni-NTA) metal affinity chromatography, as described the supplier (Qiagen).

Enzyme Production for CLEAs

The SPase gene from B. adolescentis LMG 10502 was recombinantly expressed in E. coli XL10-Gold, under control of the constitutive promotor P34 [De Mey et al. 2007, BCM Biotechnol.:34]. Transformed cells were cultivated in 1 1 shake flasks at 37° C. using LB medium supplemented with 100 mg 1⁻¹ ampicillin. After 8 h of expression, the cells were harvested by centrifugation (7000 rpm, 4° C., 20 min) Crude enzyme preparations were prepared by enzymatic lysis of frozen pellets using the EasyLyse Bacterial Protein Extraction Solution (Epicentre). Cell debris was removed by centrifugation (12000 rpm, 4° C., 30 min) The crude enzyme preparation was heat purified by incubation at 60° C. for 60 min Denaturated proteins were removed by centrifugation (12000 rpm, 4° C., 15 min).

Optimization of Immobilization Procedure on a Carrier

To optimize the immobilization process, several conditions (pH, temperature and buffer) were tested. For each condition 0.1 g Sepabeads EC-HFA were added to 5 ml of purified SPase solution (0.8 U ml⁻¹ or 5 ng ml⁻¹) and gently stirred at 150 rpm for 48 h and 22 h, respectively. The immobilizates were washed intensively with 100 mM phosphate buffer, pH 7.0. The washing steps and the supernatant after immobilization were stored to determine the amount of enzyme activity lost during the immobilization procedure. The immobilization yield (Y) was determined from the difference of the activity of the immobilized enzyme (Uᵢₘₘ) and the activity of free enzyme added (U_{free}). Actively bound enzyme yield (Y_{act}) was determined by the difference of the activity of the immobilized enzyme (Uᵢₘₘ) and the activity of free enzyme added (U_{free}) reduced by the remaining activity in the supernatant (U_{SN}) and non-covalently bound enzyme in washing buffer (U_{wash}).

In all cases, a reference suspension was prepared having exactly the same enzyme concentration and conditions, adding instead of the support the corresponding amount of inert wet-agarose. In all cases, the activity of this reference was fully preserved during immobilization.

Blocking of the Remaining Epoxy-Groups

At the end of the immobilization process, the immobilized support was treated for 24 h with 5 ml of 3 M glycine solution at pH 8.5 (25° C.) in order to inactivate the remaining epoxy-groups and stabilize the immobilized enzyme (Mateo et al., 2003). After this treatment, the immobilized SPase was then washed with an excess of 100 mM phosphate buffer to eliminate proteins non-covalently linked to the carrier.

Loading Capacity of Sepabeads EC-HFA

Different amount of SPase, up to 260 U, were offered to 0.1 g support in 5 ml of the optimal buffer. Y and Y_{act} were determined.

CLEAs Production

Aggregates of SPase were prepared by adding 6 ml of tent-butyl alcohol under agitation to 4 ml of heat-purified enzyme at pH 7. After 30 min, a glutaraldehyde solution of varying concentrations was added (25% v/v) to cross-link the enzyme aggregate, and the mixture was kept under stiffing for 15, 30, 60 or 120 min. Reduction of the formed imine bond was achieved by adding 10 ml of a solution containing 1 mg ml⁻¹ sodium borohydride in 0.1 M sodium bicarbonate buffer at pH 10. After 15 min, another 10 ml was added and allowed to react for 15 min. Finally, the CLEAs were separated by centrifugation (15 min at 12000 rpm) and washed five times with 0.1 M phosphate buffer at pH 7. All the steps were performed in a thermoshaker (Eppendorf) at 750 rpm and 4° C. The immobilization yield is defined as the ratio of the activity detected in the CLEA preparation and that present in the original enzyme solution.

Free Enzyme Activity Assay

Two methods were used to determine the free SPase activity (37° C.). Mostly described is the continuous coupled enzymatic assay, in which production of α-D-G1P from sucrose and inorganic phosphate is coupled to the reduction of NAD⁻ in presence of phosphoglucomutase (PGM) and glucose-6-phosphate dehydrogenase (G6P-DH) (Koga et al., 1991; Silverstein et al., 1967). The assay solution contained 50 mM Tris buffer, pH 7.0, 1 mM EDTA, 5 mM MgSO₄, 1 mM β-NAD, 5 μM G-1,6-PP, 0.6 U PGM and 0.6 U G6P-DH final concentration. The substrate was composed of 100 mM sucrose in 100 mM phosphate buffer pH 7.0 as final concentrations. The increase in absorbance at 340 nm was recorded in a spectrophotometer equilibrated at 37° C. One unit of SPase activity was defined as the amount of enzyme that released 1 μmol α-D-G1P min⁻¹.

The second method that was used, was the discontinuous Bicinchonic Acid (BCA) assay. One unit of SPase activity was expressed in terms of reducing sugar release as measured by the BCA method (Waffenschmidt and Jaenicke, 1987). Free enzyme was incubated at 37° C. in 100 mM sucrose and 100 mM phosphate buffer, pH 7.0. At certain times, samples were taken and inactivated by heating, for measuring reducing sugar release by the BCA assay.

Protein concentration was measured according to the BCA Protein assay (Pierce), with bovine serum albumin as a standard.

Immobilized Enzyme Activity Assay

The activity of immobilized SPase was determined by adding the total amount of washed immobilized enzyme (0.1 g) into 40 ml substrate solution composed of 100 mM sucrose and 100 mM phosphate buffer pH 7.0. The mixture was incubated in a thermoshaker (Eppendorf) with constant shaking (750 rpm) at 37° C. At certain times, samples were taken and inactivated by heating, for measuring reducing sugar release by BCA assay.

CLEAs Activity Assays

The phosphorolytic activity of SPase was determined by measuring the release of the reducing sugar fructose from the non-reducing substrate sucrose with the bicinchonic acid (BCA) method [Waffenschmidt and Jaenicke, 1987]. The reactions were analysed in a discontinuous way, by inactivation samples (5 min at 95° C.) at regular intervals. One unit (U) of SPase activity corresponds to the release of 1 μmole fructose from 100 mM sucrose in 100 mM phosphate buffer at pH 7 and 37° C. To determine phosphatase activity, the samples were also analysed for the release of glucose (from the α-glucose-1-phosphate generated by SPase) with the glucose oxidase/peroxidase assay [Werner et al. 1970, Z. Anal. Chem: 224]. One unit (U) of phosphatase activity corresponds to the release of 1 μmole of glucose from 100 mM sucrose in 100 mM phosphate buffer at pH 7 and 37° C. When phosphatase activity was detected, this was subtracted from the values obtained by the BCA-method to calculate the net SPase activity. The protein concentration was measured with the Protein Assay kit from Pierce, using bovine serum albumin as standard.

Determination of Optimum pH and Temperature

The influence of pH on free and immobilized enzyme was studied between 4.5 and 8.0. Enzyme activity was measured with the BCA assay at 37° C. in 100 mM phosphate buffer.

Thermoactivity of the free and immobilized enzyme were compared over the range of 30-70° C. and 30-80° C., respectively, in 100 mM phosphate buffer pH 7.0 by the BCA assay.

Kinetic Parameters for Sucrose

Initial rate measurements were carried out at optimum temperature in 100 mM phosphate buffer at optimum pH using a discontinuous assay. Apparent kinetic parameters for the phosphorolysis direction were determined by measuring the release of α-D-G1P and reducing sugar. Sucrose concentrations were varied in the range of 1.5-40 mM, while the concentration of phosphate was kept constant at a saturating concentration of approximately 5-10 times the apparent Kₘ value. The kinetic parameters were obtained from non-linear fits of the Michaelis-Menten equation to the initial rates.

Thermal Stability Assays

Free and immobilized enzyme were placed in 100 mM phosphate buffer pH 7.0 and incubated at different temperatures in a Thermoblock (Stuart SBH130D). At certain times, samples were taken and the remaining activity was determined using the BCA method.

The influence of the SPase concentration on thermal stability was determined by inactivating different concentration of enzyme.

Stability Assays of CLEAs

To determine the thermostability of SPase, soluble or immobilized enzyme was incubated in 100 mM phosphate buffer pH 7 in a water bath at 60° C. At regular intervals, samples were inactivated and the residual activity was analysed using the BCA method. To evaluate the reusability of SPase CLEAs, the biocatalyst was used for several reaction cycles of 1 h at 60° C. The enzyme was recuperated by centrifugation (15 min at 12000 rpm) and washed five times with 0.1 M phosphate buffer at pH 7.

Experimental Design and Data Analysis

All statistical analyses were performed with the statistical software environment R (Gentleman & Ihaka, 1997).

Results

Production and Purification of Recombinant His-Tagged SPase (Carrier)

The SPase gene from B. adolescentis LMG 10502 was cloned into a pCXhP34 vector and recombinantly expressed in E. coli XL10-Gold as described previously (Aerts et al., 2010). After chemo-enzymatic cell lysis, a crude enzyme preparation was obtained with a specific SPase activity of approximately 29 U mg⁻¹.

The recombinant enzyme carries a N-terminal fusion peptide with the sequence Gly-Gly-Ser-His₆-Gly-Met-Ala-Ser that provides metal-binding affinity. SPase could therefore be purified by affinity chromatography on a nickel-nitrilotriacetic acid (Ni-NTA) metal matrix. The purified biocatalyst migrated as a single protein band in Coomassie-stained SDS-PAGE. After buffer exchange in a centricon, a purification yield of 45% and a 5.5 fold increase in specific activity (to 161 U mg⁻¹) could be determined.

Characterisation of Recombinant His-Tagged SPase

Assays of the phosphorolytic activity of His₆-SPase revealed an optimal pH and temperature of 6.5 and 58° C., respectively.

The kinetic parameters K_{M} and k_{cat} for sucrose have been found to be 6.8±1.2 mM and 207±17 s⁻¹, respectively, at 58° C. and pH 6.5.

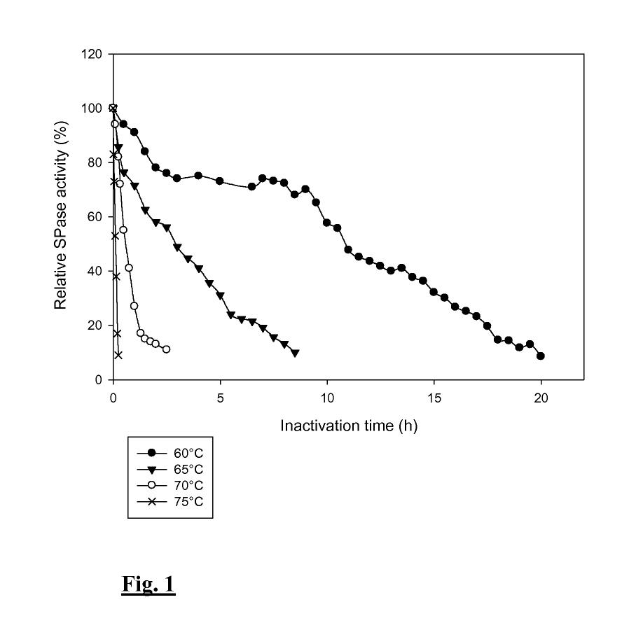

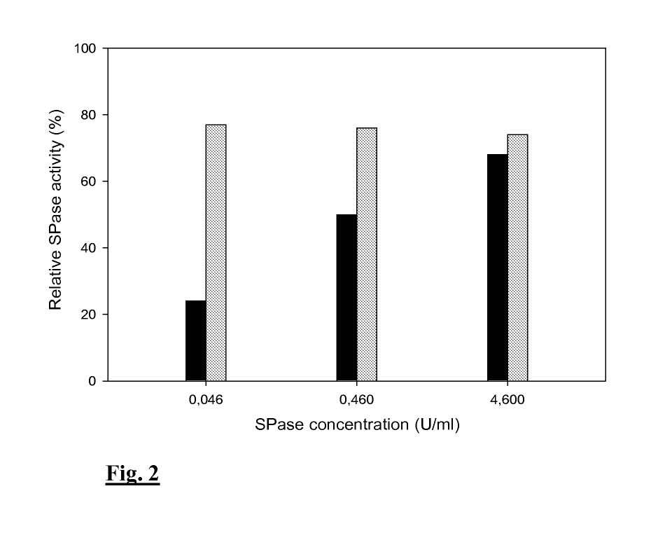

Because of its high optimum temperature, it was also interesting to examine the thermostability of the enzyme. Initial experiments with the crude enzyme preparation indicated that SPase retained more than 70% of the initial activity when incubated in phosphate buffer pH 6.5 for 30 min at 70° C. In contrast, the purified enzyme preparation (0.46 U ml⁻¹) only retained 50% of its activity under those conditions (FIG. 1). This difference suggests that the SPase may be stabilized by effectors present in the crude enzyme preparation. Furthermore, the stability of the purified SPase has been found to strongly depend on the enzyme concentration, while this is not the case for the crude enzyme preparation (FIG. 2).

Optimization of the Immobilization Process

Many factors can influence the efficiency of an immobilization process, including the temperature, pH and ionic strength of the immobilization buffer (Clark, D. S., Trends Biotechnol. 1994: 439). Here, the effect of these parameters on the immobilization of SPase on sepabeads EC-HFA was investigated by full factorial design. A phosphate buffer was used in all experiments, and its strength was varied by changing the phosphate concentration. A scree plot revealed that the temperature did not influence the immobilization yield and this parameter was, therefore, set at a fixed value of 25° C. in all consecutive experiments. The influence of the independent variables pH and phosphate concentration was further evaluated according to central composite design (CCD), using a generalized linear model. The optimal values were found to be a pH of 7.15 and a phosphate concentration of 0.04 M, resulting in a predicted immobilization yield of 71.9%.

Evaluation of the Immobilization Process

When immobilizing SPase on Sepabeads EC-HFA at optimal conditions, total adsorption of the enzyme was achieved within 22 h (complete loss of activity in the supernatant). At 25 ng protein per gram support (40 U g⁻¹ support), a recovery of 28 U g⁻¹ was obtained (yield of 70%). About 10% of the enzyme was removed during the washing steps and thus non-covalently bound, so it can be assumed that the immobilized enzyme loses approximately 22% of its activity due to a suboptimal conformation and/or to diffusional problems. The loss of activity during the washing steps could not be avoided by increasing the immobilization time.

The presence of 500 mM sucrose during immobilization on Sepabeads EC-HFA did not influence the efficiency of the immobilization. Blocking the free epoxy-groups at the end of the reaction also did not influence the immobilization efficiency (Mateo et al., 2003)

Loading Capacity of Sepabeads

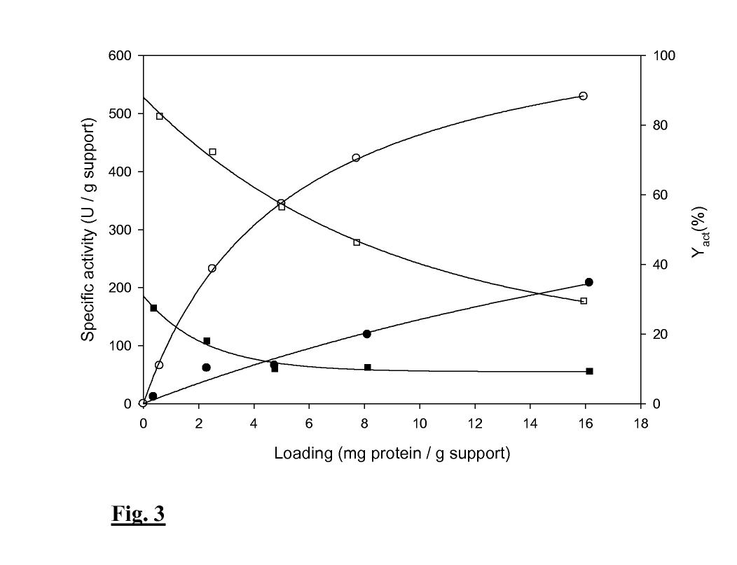

In order to determine the maximum loading capacity of Sepabeads EC-HFA, different amounts of enzyme (with a specific activity of 161 U mg⁻¹) were offered for immobilization. In each case, the activity of the supernatant, the washing buffer and the immobilised biocatalyst was determined (FIG. 3). When offering 16 mg protein per gram Sepabeads EC-HFA, maximum loading capacity was all but reached, leading to approximately 530 U g⁻¹ support. However, this came at a high price: 35% of bound enzyme was lost during the washing steps, and only 30% of covalently bound enzyme was active. Most likely, diffusional problems worsen when higher amounts of enzyme are bound.

Properties of Immobilized Enzyme

Studies have shown that the activity and stability of an immobilized enzyme, might or might not differ from those of its soluble counterpart (Clark, 1994; Mateo et al., 2007). In the present invention, the properties of the SPase immobilized on Sepabeads, in particular on Sepabeads EC-HFA, were compared with those of the free enzyme.

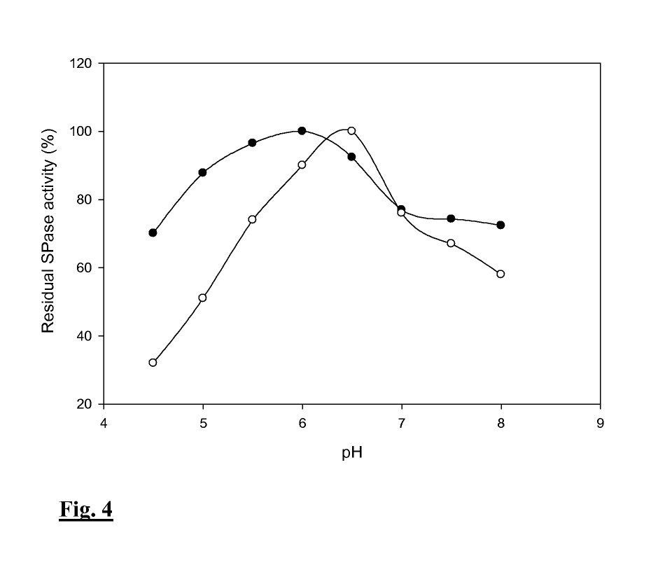

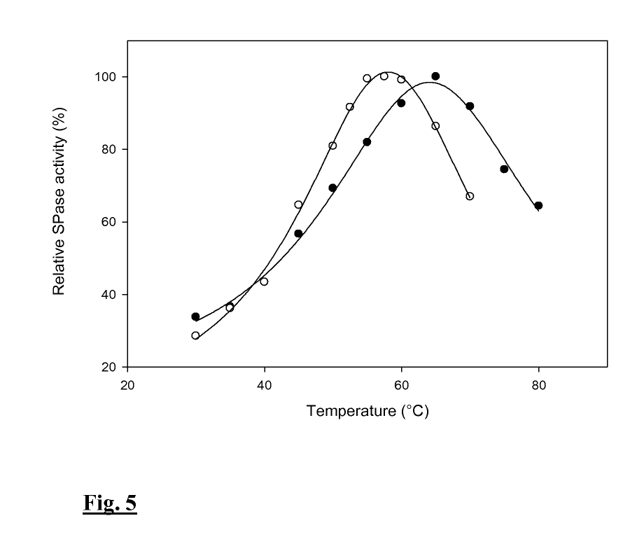

The optimal pH and temperature for activity of the immobilized enzyme were found to be 6.0 and 65° C., respectively, compared to 6.5 and 58° C. for the free enzyme (FIGS. 4 and 5). Furthermore, the immobilized enzyme is active in a broader pH-range, indicating a higher operational stability. An evaluation of the influence on thermostability is, however, less straightforward. The immobilized SPase retains 65% of its activity after 16 h incubation at 60° C., while this varies for the free enzyme according to its concentration. The maximal residual activity that could be reached is 80%. This, however, requires an enzyme concentration of 20 U ml⁻¹, which is not very realistic in practice. In contrast, the stability of the immobilized enzyme is constant in the range of 0-800 U g⁻¹. Furthermore, its residual activity could further be increased to 75% by immobilization in the presence of sucrose (500 mM), which does not influence the immobilization yield.

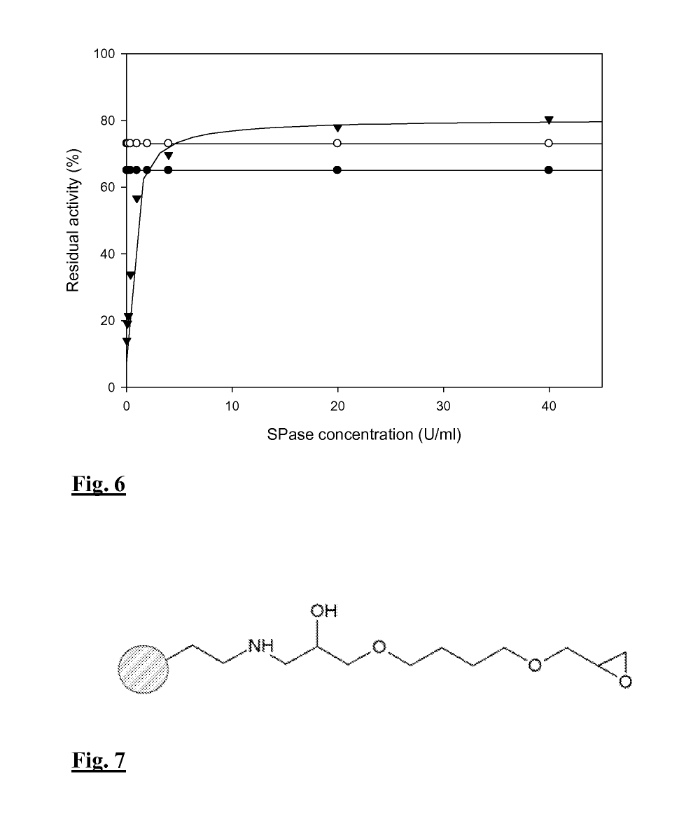

Finally, the kinetic parameters of the immobilized SPase were determined at 65° C. and pH 6.0. The k_{cat} and K_{M} for sucrose were found to be 310±24 s⁻¹ and 9.4±1.3 mM, respectively. The K_{M} of the immobilized SPase is slightly higher than that of the free enzyme, suggesting that immobilization causes diffusional restrictions. The k_{cat} value, in contrast, is higher than that of the free enzyme. Therefore, the loss of activity caused by immobilization is compensated by the higher substrate turn-over that is achieved at the higher optimum temperature (65° C. compared to 58° C.) (FIG. 6).

Operational Stability of the Free Enzyme in a Conversion Process

A conversion reaction at 60° C. was performed with a crude enzyme preparation of SPase, to determine the enzyme's operational stability in the presence of substrate. One unit of SPase was mixed with 40 ml of substrate solution, containing 100 mM sucrose in 100 mM phosphate buffer pH 7. The reaction rate was found to be constant up to 24 h, indicating that the enzyme is stabile under these process conditions.

Continuous Process in a Fixed-Bed Reactor Using SP Immobilized on Sepabeads

A continuous process was carried out in a packed-bed reactor containing SPase immobilised on Sepabeads EC-HFA. A substrate solution of 400 mM sucrose in 400 mM phosphate at pH 7 and 60° C. was pumped through the column at a flow rate of 0.75 ml min⁻¹, corresponding to a residence time of 24 min. A degree of conversion of 69% could be achieved, corresponding to a productivity of 179.5 gl⁻¹ h⁻¹. Surprisingly, the conversion rate was found to remain constant up to 2 weeks, emphasizing the remarkable operational stability of the immobilized SPase.

Production and Purification of SPase (CLEA's)

The SPase gene from B. adolescentis LMG 10502 was recombinantly expressed in E. coli XL10-Gold. After chemo-enzymatic cell lysis, a crude enzyme preparation was obtained with a specific SPase activity of approximately 13 U mg⁻¹ at 37° C. As the SPase is more stable than most endogenous E. coli proteins, the enzyme could be partially purified by means of heat treatment (Table 1). In this way, all phosphatase activity was removed, which would otherwise degrade the α-glucose-1-phosphate (G1P) produced by SPase.

After 1 hour incubation at 60° C., the specific activity increases to 29 U mg⁻¹ and the concentration of soluble protein drops from 2.6 to 1.2 mg ml⁻¹. The latter is completely due to the loss of contaminating proteins, as no decrease in SPase activity is observed under these conditions. Longer incubation times do not result in a further increase in specific activity.

TABLE 1
Heat purification of SPase from B. adolescentis

| Incubation | SPase | Phosphatase | Protein | |
|---|---|---|---|---|
| at 60° C. | activity | activity | concentration | Specific activity |
| (min) | (U/ml) | (U/ml) | (mg/ml) | (U/mg) |
| 0 | 35.1 | 2.7 | 2.6 | 13.5 |
| 20 | 35.2 | 0.7 | 1.6 | 22.0 |
| 40 | 35.0 | 0.0 | 1.5 | 23.3 |
| 60 | 35.1 | 0.0 | 1.2 | 29.3 |
| 90 | 35.0 | 0.0 | 1.2 | 29.2 |

Assays were performed at 37° C. using 100 mM sucrose in 100 mM phosphate buffer pH 7 as substrate. SPase activity corresponds to the release of fructose, while phosphatase activity corresponds to the release of glucose (from the α-glucose-1-phosphate formed by SPase).

Production of SPase CLEAs

The first step in the preparation of CLEAs consists of the aggregation of the enzymes, which can be achieved by the addition of salts, organic solvents or non-ionic polymers [Cao et al., 2000, Org. Lett.:1361)]. The choice of the additive is important, because it can result in enzymes with slightly different three-dimensional structures. Ammonium sulfate is the most widely used precipitant for protein purification, but gave unsatisfactory results with SPase. High concentrations of the salt are required (˜70% w/v) to aggregate this enzyme and generate a gelatinous suspension that is difficult to centrifuge. Precipitation was, therefore, performed with tert-butanol instead. A solvent concentration of 60% (v/v) resulted in complete removal of SPase activity from the supernatant after centrifugation. The precipitate could be redissolved in phosphate buffer without loss of activity, indicating that the aggregation procedure does not damage the structural integrity of the protein.

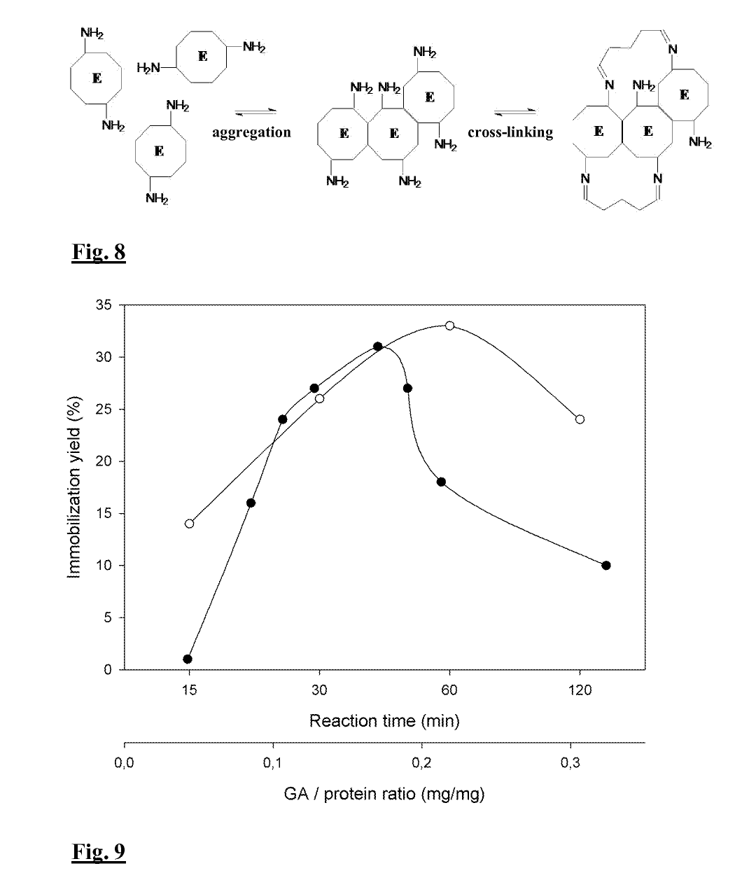

In the second step, the aggregated enzyme molecules are chemically cross-linked to obtain an immobilised biocatalyst. Glutaraldehyde (GA) is generally used for that purpose, as it contains two aldehyde groups that can form imine bonds with lysine residues from two enzyme molecules (FIG. 8). It is well known that the immobilization yield strongly depends on the incubation time of the cross-linking step as well as on the GA/protein ratio [Wilson et al., 2009, Process Biochem:322]. These parameters have, therefore, been optimized for the production of CLEAs of SPase (FIG. 9). A maximal immobilization yield of 31% could be achieved at a GA/protein ratio of 0.17 mg mg⁻¹ and an incubation time of 1 hour. Higher ratios and longer incubation times result in a considerable reduction in catalytic activity, most likely because glutaraldehyde then starts to react with residues in the active site.

Characterization of SPase CLEAs

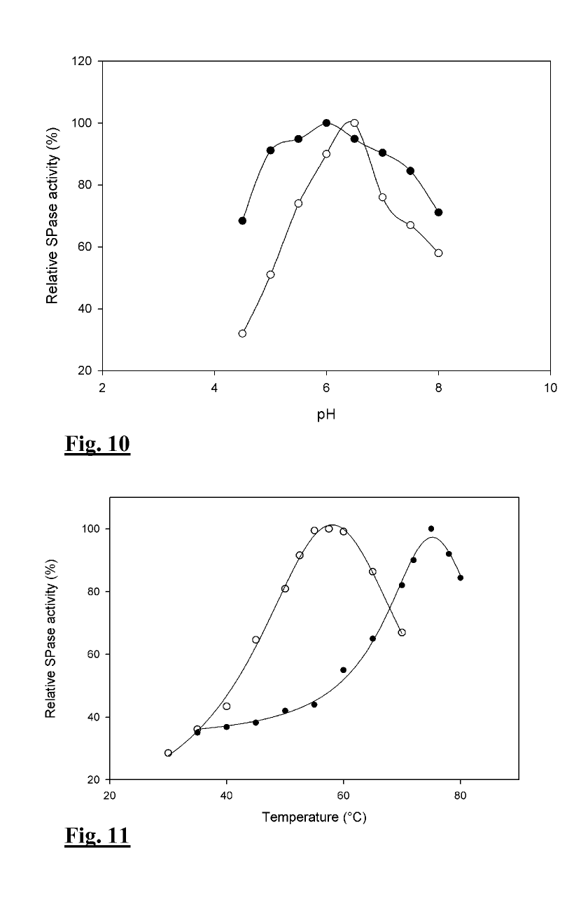

The properties of the CLEAs were compared with those of the native SPase. The optimal pH and temperature for phosphorolytic activity of the immobilized enzyme were found to be 6.0 and 75° C., compared to 6.5 and 58° C., respectively, for the soluble enzyme (FIG. 10 and FIG. 11). Cross-linking thus results in an enzyme whose temperature optimum has increased with an impressive 17° C. Furthermore, the immobilized enzyme is active in a broader pH-range, indicating a higher operational stability.

To determine the thermostability of the SPase preparation, the enzyme was incubated at 60° C. and its residual activity was measured at several points in time. The CLEAs were found to retain full activity after 1 week incubation, whereas the free enzyme looses 20% of its activity after only 16 h incubation. The stability of the biocatalyst is, therefore, dramatically improved by the cross-linking process. As industrial carbohydrate conversions are preferably performed at 60° C., the properties of these CLEAs will undoubtedly allow the development of novel processes of high economic value.

One of the major advantages of immobilization is that it leads to an enzyme preparation that can be recycled, which often is a key determinant of its industrial potential. CLEAs can be easily recycled by either filtration or centrifugation [Cao et al. 2003: Curr. Opin. Biotechnol: 387] as has been used in the present invention. Centrifugation at high speeds (12000 rpm) was found to be required for the precipitation of CLEAs and the complete removal of phosphorolytic activity from the supernatant. To evaluate the mechanical stability of the biocatalyst under these conditions, several reaction cycles of 1 h at 60° C. were performed with thorough washing in between. After ten cycles, no loss of activity could be detected, revealing the excellent operational stability of the new enzyme preparation.

Production of G1P with SPase CLEAs

To evaluate the efficiency of the SPase CLEAs in a production process, the phosphorolysis of sucrose into G1P was monitored at 60° C. and pH 7 until maximal conversion. The reaction was performed with 500 U of CLEAs in a solution containing 1 M of sucrose and inorganic phosphate. After about 20 h, the conversion was finished and 0.7 M G1P was produced. This corresponds to an equilibrium constant (K_{eq}) of 5.63, which is slightly higher than that of other glycoside phosphorylases [Nidetzky et al. 2000, Biochel J.: 649].

In view of the exceptional mechanical and thermal stability of the CLEAs, this reaction can be repeated at least seven times in one week time. In that way, more than 1 kg of G1P is produced with only about 50 mg of protein, which still would be fully active. This is the first report on a production process with SPase at elevated temperatures.

### Example 2

### Mutating the SPase to Further Increase its Stability

Materials, Methods and Results

Mutations were introduced by High Fidelity PCR followed by digestion with DpnI (New England Biolabs). The enzyme variants were produced and purified as described for the wild-type enzyme. Their thermostability was assayed by incubating 85 μg/ml of enzyme for 16 h at 60° C., after which the residual activity was determined. The enzyme variants displayed an activity that corresponds to at least 80% of the wild-type activity, illustrating that their increased stability did not come at the expense of activity.

As can be seen in Table 2, introducing mutations R393N, Q460E/E485H, D445P/D446T or D445/D446G, or Q331E increases the enzyme's stability considerably. Furthermore, combining all of these mutations results in an enzyme variant that is completely stable for 16 h at 60° C.

TABLE 2

| Enzyme | Mutations | Residual activity (%) |
|---|---|---|
| wild-type | — | 79 |
| variant A | R393N | 81 |
| variant B | Q460E/E485H | 84 |
| variant C or C′ | D445P/D446T or D445P/D446G | 81 |
| variant D | Q331E | 84 |
| variant AB | R393N/Q460E/E485H | 87 |
| variant ABC or | R393N/Q460E/E485H/D445P/ | 90 |
| ABC′ | D446T | |
| variant ABCD | R393N/Q460E/E485H/ | 100 |
| or ABC′D | D445P/D446T/Q331E | |

### REFERENCES

Aerts, D., Verhaeghe, T., De Mey, M., Desmet, T., Soetaert, W., 2010. A constitutive expression system for high throughput screening Eng. Life Sci. 11(1): 10-19.

Alonso-Morales, N., Lopez-Gallego, F., Betancor, L., Hidalgo, A., Mateo, C., Fernandez-Lafuente, R., Guisan, J. M., 2004. Reversible immobilization of glutaryl acylase on sepabeads coated with polyethyleneimine. Biotechnol. Prog. 20, 533-536.

Alonso, N., Lopez-Gallego, F., Betancor, L., Hidalgo, A., Mateo, C., Guisan, J. M., Fernandez-Lafuente, R., 2005. Immobilization and stabilization of glutaryl acylase on aminated sepabeads supports by the glutaraldehyde crosslinking method. J. Mol. Catal. B: Enzym. 35, 57-61.

Birnberg, P. R., Brenner, M. L., 1984. A one-step enzymatic assay for sucrose with sucrose phosphorylase. Anal. Biochem. 142, 556-561.

Bolivar, J. M., Wilson, L., Ferrarotti, S. A., Fernandez-Lafuente, R., Guisan, J. M., Mateo, C., 2006. Stabilization of a formate dehydrogenase by covalent immobilization on highly activated glyoxyl-agarose supports. Biomacromolecules 7, 669-673.

Clark, D. S., 1994. Can immobilization be exploited to modify enzyme-activity. Trends Biotechnol. 12, 439-443.

Fujii, K., Liboshi, M., Yanase, M., Takaha, T., Kuriki, T., 2006. Enhancing the thermal stability of sucrose phosphorylase from Streptococcus nutans by random mutagenesis. J. Appl. Glycosci. 53, 91-97.

Goedl, C., Nidetzky, B., 2009. Sucrose phosphorylase harbouring a redesigned, glycosyltransferase-like active site exhibits retaining glucosyl transfer in the absence of a covalent intermediate. Chembiochem 10, 2333-2337.

Goedl, C., Schwarz, A., Minani, A., Nidetzky, B., 2007. Recombinant sucrose phosphorylase from Leuconostoc mesenteroides: Characterization, kinetic studies of transglucosylation, and application of immobilised enzyme for production of alpha-D-glucose 1-phosphate. J. Biotechnol. 129, 77-86.

Henrissat, B., 1991. A classification of glycosyl hydrolases based on amino-acid-sequence similarities. Biochem. J. 280, 309-316.

Hilterhaus, L., Minow, B., Muller, J., Berheide, M., Quitmann, H., Katzer, M., Thum, O., Antranikian, G., Zeng, A. P., Liese, A., 2008. Practical application of different enzymes immobilized on sepabeads. Bioprocess Biosyst. Eng. 31, 163-171.

Katchalski-Katzir, E., Kraemer, D. M., 2000. Eupergit (R) C, a carrier for immobilization of enzymes of industrial potential. J. Mol. Catal. B: Enzym. 10, 157-176.

Kitao, S., Matsudo, T., Saitoh, M., Horiuchi, T., Sekine, H., 1995. Enzymatic synthesis of 2 stable (−)-epigallocatechin gallate-glucosides by sucrose phosphorylase. Biosci., Biotechnol., Biochem. 59, 2167-2169.

Koga, T., Nakamura, K., Shirokane, Y., Mizusawa, K., Kitao, S., Kikuchi, M., 1991. Purification and some properties of sucrose phosphorylase from Leuconostoc mesenteroides. Agr. Biol. Chem. 55, 1805-1810.

Lee, J. H., Yoon, S. H., Nam, S. H., Moon, Y. H., Moon, Y. Y., Kim, D., 2006. Molecular cloning of a gene encoding the sucrose phosphorylase from Leuconostoc mesenteroides B-1149 and the expression in Escherichia coli. Enzyme Microb. Technol. 39, 612-620.

Lopez-Gallego, F., Betancor, L., Hidalgo, A., Alonso, N., Fernandez-Lorente, G., Guisan, J. M., Fernandez-Lafuente, R., 2005. Preparation of a robust biocatalyst of D-amino acid oxidase on sepabeads supports using the glutaraldehyde crosslinking method. Enzyme Microb. Technol. 37, 750-756.

Lopez-Gallego, F., Betancor, L., Hidalgo, A., Dellamora-Ortiz, G., Mateo, C., Fernandez-Lafuente, R., Guisan, J. M., 2007. Stabilization of different alcohol oxidases via immobilization and post immobilization techniques. Enzyme Microb. Technol. 40, 278-284.

Mateo, C., Abian, O., Fernandez-Lorente, G., Pedroche, J., Fernandez-Lafuente, R., Guisan, J. M., 2002. Epoxy sepabeads: A novel epoxy support for stabilization of industrial enzymes via very intense multipoint covalent attachment. Biotechnol. Prog. 18, 629-634.

Mateo, C., Archelas, A., Fernandez-Lafuente, R., Guisan, J. M., Furstoss, R., 2003. Enzymatic transformations Immobilized A-niger epoxide hydrolase as a novel biocatalytic tool for repeated-batch hydrolytic kinetic resolution of epoxides. Organic & Biomolecular Chemistry 1, 2739-2743.

Mateo, C., Grazu, V., Pessela, B. C. C., Montes, T., Palomo, J. M., Torres, R., Lopez-Gallego, F., Fernandez-Lafuente, R., Guisan, J. M., 2007. Advances in the design of new epoxy supports for enzyme immobilization-stabilization. Biochem. Soc. Trans. 35, 1593-1601.

Sheldon R A, Schoevaart R, van Langen I M (2005) Crosslinked enzyme aggregates (CLEAs): a novel and versatile method for enzyme immobilization (a review). Biocatal Biotransformation 23:141-147

Segura, R. L., Palomo, J. M., Mateo, C., Cortes, A., Terreni, M., Fernandez-Lafuente, R., Guisan, J. M., 2004. Different properties of the lipases contained in porcine pancreatic lipase extracts as enantioselective biocatalysts. Biotechnol. Prog. 20, 825-829.

Silverstein, R., Voet, J., Reed, D., Abeles, R. H., 1967. Purification and mechanism of action of sucrose phosphorylase. J. Biol. Chem. 242, 1338-1346.

Sprogoe, D., van den Broek, L. A. M., Mirza, O., Kastrup, J. S., Voragen, A. G. J., Gajhede, M., Skov, L. K., 2004. Crystal structure of sucrose phosphorylase from Bifidobacterium adolescentis. Biochemistry 43, 1156-1162.

Tedokon, M., Suzuki, K., Kayamori, Y., Fujita, S., Katayama, Y., 1992. Enzymatic assay of inorganic phosphate with use of sucrose phosphorylase and phosphoglucomutase. Clin. Chem. 38, 512-515.

Torres, R., Mateo, C., Fernandez-Lorente, G., Ortiz, C., Fuentes, M., Palomo, J. M., Guisan, J. M., Fernandez-Lafuente, R., 2003. A novel heterofunctional epoxy-amino sepabeads for a new enzyme immobilization protocol: Immobilization-stabilization of beta-galactosidase from Aspergillus oryzae. Biotechnol. Prog. 19, 1056-1060.

Torres, R., Pessela, B. C. C., Mateo, C., Ortiz, C., Fuentes, M., Guisan, J. M., Fernandez-Lafuente, R., 2004. Reversible immobilization of glucoamylase by ionic adsorption on sepabeads coated with polyethyleneimine. Biotechnol. Prog. 20, 1297-1300.

Unsworth, L. D., van der Oost, J., Koutsopoulos, S., 2007. Hyperthermophilic enzymes—stability, activity and implementation strategies for high temperature applications. Febs J. 274, 4044-4056.

van den Broek, L. A. M., van Boxtel, E. L., Kievit, R. P., Verhoef, R., Beldman, G., Voragen, A. G. J., 2004. Physico-chemical and transglucosylation properties of recombinant sucrose phosphorylase from Bifidobacterium adolescentis DSM20083. Appl. Microbiol. Biotechnol. 65, 219-227.

Waffenschmidt, S., Jaenicke, L., 1987. Assay of reducing sugars in the nanomole range with 2,2′-bicinchoninate. Anal. Biochem. 165, 337-340.

Weinhausel, A., Griessler, R., Krebs, A., Zipper, P., Haltrich, D., Kulbe, K. D., Nidetzky, B., 1997. alpha-1,4-D-glucan phosphorylase of gram-positive Corynebacterium callunae: isolation, biochemical properties and molecular shape of the enzyme from solution X-ray scattering. Biochem. J. 326, 773-783.

1. A biocatalyst comprising a sucrose phosphorylase from Bifidobacterium adolescentis characterized in that said sucrose phosphorylase is enzymatically active for a time period of at least 16 h at a temperature of at least 60° C.

2. The biocatalyst according to claim 1, wherein said sucrose phosphorylase is immobilized, and/or is mutated and/or is in the continuous presence of its substrate.

3. The biocatalyst according to claim 1, wherein said sucrose phosphorylase: a) is immobilized on an epoxy-activated enzyme carrier, or b) is part of a cross-linked enzyme aggregate (CLEA).

4. The biocatalyst according to claim 1, wherein said sucrose phosphorylase is encoded by the sucrose phosphorylase gene from Bifidobacterium adolescentis LMG 10502.

5. The biocatalyst according to claim 1, wherein said sucrose phosphorylase comprises at least one deletion, substitution or addition, or any combination thereof, which does not diminish the sucrose phosphorylase activity by at most 50%.

6. The biocatalyst according to claim 5, wherein said sucrose phosphorylase comprises the following mutations: Q331E, R393N, Q460E/E485H, D445P/D446G, D445P/D446T, R393N/Q460E/E485H, R393N/Q460E/E485H/D445P/D446T, and/or R393N/Q460E/E485H/D445P/D446T/Q331E.

7. The biocatalyst according to claim 3, wherein said epoxy-activated enzyme carrier comprises the following structure: R—O—R′, wherein R is a highly porous methacrylic polymer matrix and R′ is an epoxy group.

8. The biocatalyst according to claim 2, wherein said substrate is sucrose.

9. A method to convert carbohydrates comprising contacting a carbohydrate substrate with the biocatalyst of claim 1 at a temperature of at least 60° C.

10. The method according to claim 9, wherein said temperature is at least 65° C.

11. The method according to claim 9, wherein said conversion of carbohydrates is the phosphorolysis of sucrose into alpha-D-glucose-1-phosphate and fructose.

12. A method to produce a biocatalyst according to claim 1 comprising:
purifying a sucrose phosphorylase from Bifidobacterium adolescentis to obtain a solution of purified sucrose phosphorylase,
adding an epoxy-activated carrier to said solution of purified sucrose phosphorylase to obtain an immobilized sucrose phosphorylase,
washing said immobilized sucrose phosphorylase,
inactivating the remaining epoxy-groups on said epoxy-activated carrier which are not linked to a sucrose phosphorylase, and
washing said immobilized sucrose phosphorylase.

13. The method according to claim 12 preceded by transforming a host organism with a gene encoding for the sucrose phosphorylase from Bifidobacterium adolescentis and expressing said phosphorylase in said host organism.

14. The method according to claim 12, wherein the step of adding an epoxy-activated carrier to the solution of purified sucrose phosphorylase is undertaken in an immobilization buffer having a pH of 7.15 and containing a phosphate concentration of 0.04 M.

15. A method to produce the biocatalyst according to claim 1 comprising the following steps:
purifying a sucrose phosphorylase from Bifidobacterium adolescentis in order to obtain a solution of purified sucrose phosphorylase,
aggregating said purified sucrose phosphorylase solutionen by the addition of salts, organic solvents or non-ionic polymers to obtain aggregated sucrose phosphorylase,
chemically cross-linking said aggregated sucrose phosphorylase to obtain an immobilised biocatalyst.


SEQUENCE LISTING (2 sequences)

SEQ ID NO: 1 - protein, 504 aa, Bifidobacterium adolescentis [sequence omitted]

SEQ ID NO: 2 - protein, 13 aa, Artificial
  His-Tag
GGSHHHHHHGMAS